(19) United States
(12) Patent Application Publication
(10) Pub. No.: US 2026/0162835 A1
(43) Pub. Date: Jun. 11, 2026

(54) Veterans Assistance Software Application Device

(71) Applicant: Audrey Atkinson, Salisbury, NC (US)

(72) Inventor: Audrey Atkinson, Salisbury, NC (US)

(21) Appl. No.: 19/283,533

(22) Filed: Jul. 29, 2025

Related U.S. Application Data
(60) Provisional application No. 63/730,128, filed on Dec. 10, 2024.

(51) Int. Cl. G16H 80/00; G06Q 30/0241; G16H 20/70; G16H 40/67

(52) U.S. Cl. CPC G16H 80/00; G06Q 30/0277; G16H 20/70; G16H 40/67

(57) ABSTRACT

A veterans assistance application device is disclosed, which is a software application for assisting veterans in building a network to combat veteran mental health issues. The veterans assistance application device is a unique software application accessible on a smartphone, tablet, laptop, or a desktop computer. The application allows veterans to communicate and assist one another in dealing with their issues. The application allows veterans to reconnect and communicate with others from deployments or those experiencing similar mental health issues. The application also includes a section for advertisements and services.

### CROSS-REFERENCE TO RELATED APPLICATION

The present application claims priority to, and the benefit of, U.S. Provisional Application No. 63/730,128, which was filed on Dec. 10, 2024, and is incorporated herein by reference in its entirety.

### FIELD OF THE INVENTION

The present invention relates generally to the field of veterans assistance application devices. More specifically, the present invention relates to a software application that helps veterans build networks to combat mental health issues like depression, anxiety, and even suicide. Accordingly, the present disclosure makes specific reference thereto. Nonetheless, it is to be appreciated that aspects of the present invention are also equally applicable to other like applications, devices, and methods of manufacture.

### BACKGROUND

By way of background, this invention relates to improvements in veterans assistance application devices. Generally, military veterans may have trouble reacclimating to society after their deployment. Many military veterans suffer from PTSD and several other mental health ailments that cause them to be isolated from friends, family, and other people. People may not know where to seek help and therapy for their issues, or think that people that are not in the military may not understand their plights.

Further, searching online may lead to more questions than answers. Accordingly, a software application is needed, which would allow veterans to search easily and quickly for veterans assistance, such as, but not limited to, mental health issues, counseling, networking with other veterans, and creating lines of open communication, etc. The software application would allow owners to easily assist veterans in building a network to combat suicide, depression, and other mental health issues, schedule appointments, find doctors or other veterans, etc. Thus, the software application takes the stress and difficulty out of finding help for mental health ailments.

Accordingly, there is a demand for an improved veterans assistance application device that provides a software application for helping veterans build networks to combat mental health issues. More particularly, there is a demand for a veterans assistance application device that is a software application which allows veterans to communicate and assist one another in dealing with mental health issues.

Therefore, there exists a long-felt need in the art for a veterans assistance application device that provides users with a software application that helps veterans build networks to combat mental health issues. There is also a long-felt need in the art for a veterans assistance application device that allows veterans to communicate and assist one another in dealing with mental health issues outside of therapy sessions. Further, there is a long-felt need in the art for a veterans assistance application device that enables veterans to reconnect with one another from deployments. Moreover, there is a long-felt need in the art for a device that allows veterans to connect with others experiencing similar mental health issues. Further, there is a long-felt need in the art for a veterans assistance application device that includes a section for advertisements and services to earn money for the application. Finally, there is a long-felt need in the art for a veterans assistance application device that is available as a software application or a website platform.

The subject matter disclosed and claimed herein, in one embodiment thereof, comprises a veterans assistance application device. The device is a software application for assisting veterans in building a network to combat veteran mental health issues. The veterans assistance application device is a unique software application accessible on a smartphone, tablet, laptop, or desktop computer. The application allows veterans to communicate and assist one another in dealing with their issues. The application allows veterans to reconnect and communicate with others from deployments or those experiencing similar mental health issues. The application also includes a section for advertisements and services.

In this manner, the veterans assistance application device of the present invention accomplishes all of the foregoing objectives and provides users with a device that allows a user to build networks to combat mental health issues. The device is a software application that allows scheduling, connection, networking, and contact options. The device can be accessed through a smartphone, tablet, or a desktop computer.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a veterans assistance application device. The device is a software application for assisting veterans in building a network to combat veteran mental health issues. The veterans assistance application device is a unique software application accessible on an electronic device. The application allows veterans to communicate and assist one another in dealing with their issues.

In one embodiment, the veterans assistance application device utilizes a smartphone, a tablet, a laptop, or a desktop computer, etc., or any other suitable electronic device that has the required operating and memory parameters to support the software application of the present invention.

In one embodiment, the software application helps veterans build networks to combat mental health issues, such as depression, anxiety, suicide, etc., or any other suitable mental health issues as is known in the art. The software application allows veterans to communicate and assist one another in dealing with their mental health issues outside of therapy sessions. Specifically, the software application enables veterans to reconnect and communicate with other veterans from previous deployments or connect with veterans experiencing similar mental health issues.

In one embodiment, once downloaded, the software application allows a user to create a secure account login to protect their account settings and profile information. The secure account login prevents unauthorized users from going into the software application and acquiring user data and information. Further, business owners advertising their goods/services on the software application would also create a secure account login to protect their account settings and profile information. The secure account login prevents unauthorized users from gaining access to the software application and accessing private user and/or business data and information.

In one embodiment, the software application allows business owners to create a profile, then create an advertisement (Ad) offering for sale their services and/or goods. The business owner can then create a payment page disclosing pricing and fees for particular services and/or goods.

In one embodiment, the software application allows a user (i.e., a veteran) to create a profile, as well. The veteran profile prompts the user to upload identification, such as, but not limited to, a current ID and a military ID. Specifically, the veteran uploads the front and back of the current ID and uploads the front and back of the military ID. The current ID can be a driver's license, a passport, and identification card, etc., or any other personal ID as is known in the art. The military ID can be a common access card (CAC), which is issued to Active-duty service members, selected reserve, DOD civilian employees, and eligible personnel. Dependents, retired service members, and members of the individual ready reserve receive the Uniformed Services ID (USID) card, etc., or any other suitable military ID card as is known in the art.

Once the user creates their profile, the top of the profile will have a medical cross and a map icon of the United States of America. Tapping the map icon displays a larger map, disclosing all fifty states of the United States of America. Users can click on a specific state to view that state's resources and available mental health services, etc. Once the specific state is clicked on, the software application opens up two tabs. Specifically, each state will have a veterans tab and a business tab.

In one embodiment, the veterans tab will display active veterans. Veterans can then click on a specific veteran they wish to contact and/or talk to. Clicking on the specific veteran allows the user to view the chosen veteran's profile. After clicking the profile button, users are able to view chat, phone, or camera icons as options to communicate. The user can then contact the chosen veteran via any suitable method disclosed (i.e., chat, phone, or camera).

In one embodiment, the business tab will display all businesses with veteran offers. Veterans can then click on a specific business they are interested in. Clicking on the specific business will pull up their advertisement, disclosing their offered goods and/or services. After clicking the profile button, users are able to view chat, phone, or camera icons as options to communicate. The user can then contact the chosen business via any suitable method disclosed (i.e., chat, phone, or camera).

In one embodiment, the software application provides a secure website and/or smartphone app access by users for building networks to combat mental health issues for veterans. In addition to these elements, the device of the present invention may also include: a web server and Cloud database of veteran and business identification and data connectable to the mobile smartphone or portable tablet, or computer device, etc.

In one embodiment of the invention, the veteran and business information and data, as well as other data is maintained on a Microsoft Cloud server in a typical fashion, or other cloud server, such as the Adobe, Apple, or Amazon cloud server, etc. A secure cloud server is used for the protection and security of private veteran and business information and account information, including payment information, and other information data stored on the cloud server. Conveniently, the data is stored and managed with a data management software or program to manage the collected veteran and business information, account information, payment information, and charge history, etc.

In one embodiment, users (i.e., veterans) can obtain a subscription to the software application, which allows them to communicate directly with the listed veterans and businesses and have full access to all of the software application's services, features, and functionality. Further, the business owners will typically pay a monthly fee for use of the software application to list their business goods and/or services. In one embodiment, the software application can include advertisements and services to earn money for the software application. Thus, the veterans assistance application device allows users (i.e., veterans) to efficiently and easily communicate and assist one another in dealing with their mental issues.

In yet another embodiment, the veterans assistance application device comprises a plurality of indicia.

In yet another embodiment, a method of building veteran networks to combat mental health issues is disclosed. The method includes the steps of providing a veterans assistance application device comprising a software application accessible on a smartphone. The method also comprises accessing the software application. Further, the method comprises communicating with veterans from previous deployments via the software application. The method also comprises communicating with veterans experiencing similar mental health issues via the software application. The method comprises assisting one another outside of therapy sessions. Finally, the method comprises providing advertisements and services to earn money for the software application.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a flowchart of one embodiment of the veterans assistance application device of the present invention in accordance with the disclosed architecture;

FIG. 2 illustrates a flowchart showing the method of building veteran networks to combat mental health issues in accordance with the disclosed architecture;

FIG. 3 is a schematic diagram illustrating one embodiment of the veterans assistance application device of the present invention in accordance with the disclosed architecture; and

FIG. 4 illustrates an exemplary user device that is used for installing the veterans assistance application device of the present invention for communication with a server system in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a veterans assistance application device that provides users with a software application that helps veterans build networks to combat mental health issues. There is also a long-felt need in the art for a veterans assistance application device that allows veterans to communicate and assist one another in dealing with mental health issues outside of therapy sessions. Further, there is a long-felt need in the art for a veterans assistance application device that enables veterans to reconnect with one another from deployments. Moreover, there is a long-felt need in the art for a device that allows veterans to connect with others experiencing similar mental health issues. Further, there is a long-felt need in the art for a veterans assistance application device that includes a section for advertisements and services to earn money for the application. Finally, there is a long-felt need in the art for a veterans assistance application device that is available as a software application or a website platform.

The present invention, in one exemplary embodiment, is a novel veterans assistance application device. The device is a software application for assisting veterans in building a network to combat veteran mental health issues. The veterans assistance application device is a unique software application accessible on a smartphone, tablet, laptop, or a desktop computer. The application allows veterans to communicate and assist one another in dealing with their issues. The application allows veterans to reconnect and communicate with others from deployments or those experiencing similar mental health issues. The application also includes a section for advertisements and services. The present invention also includes a novel method of building veteran networks to combat mental health issues. The method includes the steps of providing a veterans assistance application device comprising a software application accessible on a smartphone. The method also comprises accessing the software application. Further, the method comprises communicating with veterans from previous deployments via the software application. The method also comprises communicating with veterans experiencing similar mental health issues via the software application. The method comprises assisting one another outside of therapy sessions. Finally, the method comprises providing advertisements and services to earn money for the software application.

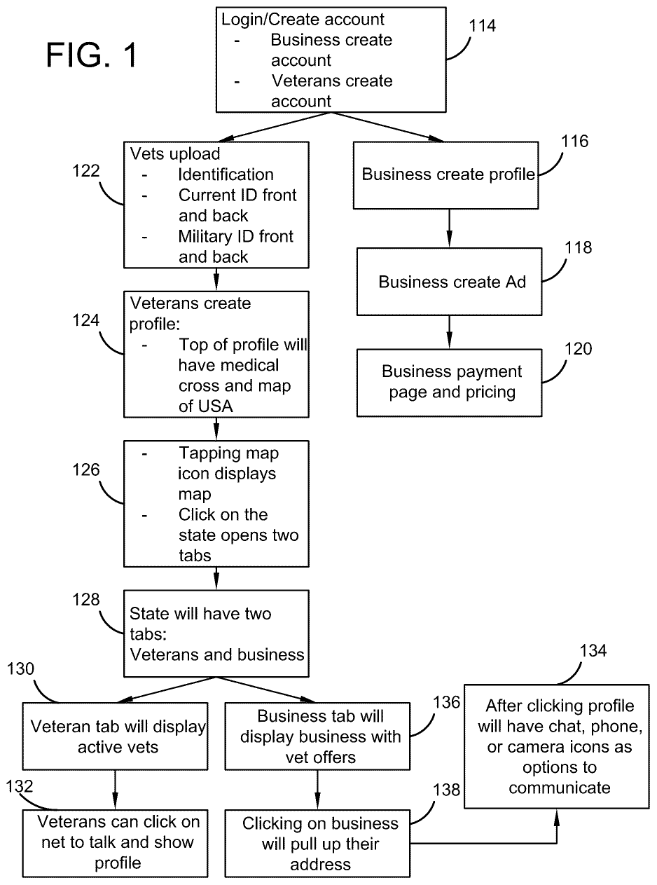

Referring initially to the drawings, FIG. 1 illustrates a perspective view of one embodiment of the veterans assistance application device 100 of the present invention. In the present embodiment, the veterans assistance application device 100 is an improved veterans assistance application device 100 that provides a user with a software application, mobile application, or website that provides networking services to combat veteran mental health issues. Specifically, the device 100 is a unique software application accessible on an electronic device, such as a smartphone or smart device (i.e., user device 102). The veterans assistance application device 100 provides a user interface to allow a user to build networks to combat mental health issues for veterans. A user would download the software application 100 and utilize the user interfaces to locate, contact, schedule, and communicate with other veterans and mental health professionals conveniently and easily.

Further, the veterans assistance application device 100 utilizes a smartphone, a tablet, a laptop, or a desktop computer, etc. (i.e., user device 102), or any other suitable electronic device that has the required operating and memory parameters to support the software application 100 of the present invention.

In one embodiment, the veterans assistance application device 100 comprises a plurality of indicia 112. In one embodiment, the veterans assistance application device 100 comprises a plurality of indicia 112. Typically, the user interface of the device 100 may include advertising, a trademark, or other letters, designs, or characters, printed, painted, stamped, or integrated into the software application, or any other indicia 112 as is known in the art. Specifically, any suitable indicia 112 as is known in the art can be included, such as, but not limited to, patterns, logos, emblems, images, symbols, designs, letters, words, characters, animals, advertisements, brands, etc., that may or may not be veterans, mental health, or brand related.

Generally, the software application 100 helps veterans build networks to combat mental health issues, such as depression, anxiety, suicide, etc., or any other suitable mental health issues as is known in the art. The software application 100 allows veterans to communicate and assist one another in dealing with their mental health issues outside of therapy sessions. Specifically, the software application 100 enables veterans to reconnect and communicate with other veterans from previous deployments or connect with veterans experiencing similar mental health issues.

Further, once downloaded, the software application 100 at 114, allows a user to create a secure account login to protect their account settings and profile information. The secure account login prevents unauthorized users from going into the software application 100 and acquiring user data and information. Further, business owners advertising their goods/services on the software application 100 would also create a secure account login to protect their account settings and profile information. The secure account login prevents unauthorized users from gaining access to the software application 100 and accessing private user and/or business data and information.

At 116, the software application allows business owners to create a profile, then at 118, create an advertisement (Ad) offering for sale their services and/or goods. At 120, the business owner can then create a payment page disclosing pricing and fees for particular services and/or goods.

At 122, the software application allows a user (i.e., a veteran) to create a profile, as well. The veteran profile prompts the user to upload identification, such as, but not limited to, a current ID and a military ID. Specifically, the veteran uploads the front and back of the current ID and uploads the front and back of the military ID. The current ID can be a driver's license, a passport, and identification card, etc., or any other personal ID as is known in the art. The military ID can be a common access card (CAC), which is issued to Active-duty service members, selected reserve, DOD civilian employees, and eligible personnel. Dependents, retired service members, and members of the individual ready reserve receive the Uniformed Services ID (USID) card, etc., or any other suitable military ID card as is known in the art.

At 124, once the user creates their profile, the top of the profile will have a medical cross and a map icon of the United States of America. At 126, tapping the map icon displays a larger map, disclosing all fifty states of the United States of America. Users can click on a specific state to view that state's resources and available mental health services, etc. At 128, once the specific state is clicked on, the software application opens up two tabs. Specifically, each state will have a veterans tab and a business tab.

At 130, the veterans tab will display active veterans. At 132, veterans can then click on a specific veteran they wish to contact and/or talk to. Clicking on the specific veteran allows the user to view the chosen veteran's profile. At 134, after clicking the profile button, users are able to view chat, phone, or camera icons as options to communicate. The user can then contact the chosen veteran via any suitable method disclosed (i.e., chat, phone, or camera).

At 136, the business tab will display all businesses with veteran offers. At 138, veterans can then click on a specific business they are interested in. Clicking on the specific business will pull up their advertisement, disclosing their offered goods and/or services. At 134, after clicking the profile button, users are able to view chat, phone, or camera icons as options to communicate. The user can then contact the chosen business via any suitable method disclosed (i.e., chat, phone, or camera).

Further, the software application 100 provides a secure website and/or smartphone app access by users for building networks to combat mental health issues for veterans. In addition to these elements, the device 100 of the present invention may also include: a web server and Cloud database of veteran and business identification and data connectable to the mobile smartphone or portable tablet, or computer device, etc., (i.e., user device 102).

Additionally, the veteran and business information and data, as well as other data, is maintained on a Microsoft Cloud server in a typical fashion, or other cloud server, such as the Adobe, Apple, or Amazon cloud server, etc. A secure cloud server is used for the protection and security of private veteran and business information and account information, including payment information, and other information data stored on the cloud server. Conveniently, the data is stored and managed with a data management software or program to manage the collected veteran and business information, account information, payment information, and charge history, etc.

Furthermore, users (i.e., veterans) can obtain a subscription to the software application 100, which allows them to communicate directly with the listed veterans and businesses and have full access to all of the software application's services, features, and functionality. Further, the business owners will typically pay a monthly fee for use of the software application 100 to list their business goods and/or services. In one embodiment, the software application 100 can include advertisements and services to earn money for the software application 100. Thus, the veterans assistance application device 100 allows users (i.e., veterans) to efficiently and easily communicate and assist one another in dealing with their mental issues.

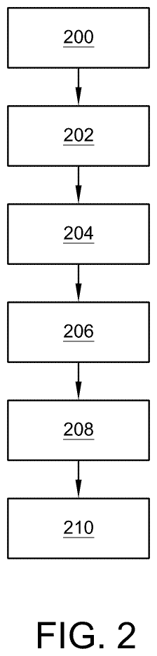

FIG. 2 illustrates a flowchart of the method of building veteran networks to combat mental health issues. The method includes the steps of at 200, providing a veterans assistance application device comprising a software application accessible on a smartphone. The method also comprises at 202, accessing the software application. Further, the method comprises at 204, communicating with veterans from previous deployments via the software application. The method also comprises at 206, communicating with veterans experiencing similar mental health issues via the software application. The method comprises at 208, assisting one another outside of therapy sessions. Finally, the method comprises at 210, providing advertisements and services to earn money for the software application.

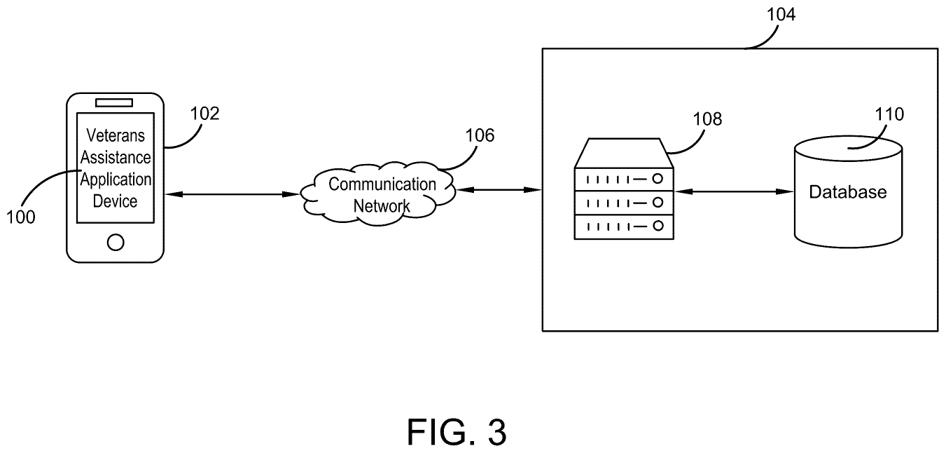

FIG. 3 is a schematic diagram illustrating one embodiment of the veterans assistance application device 100 of the present invention. The veterans assistance application device 100 has a client-server architecture wherein a user accesses the software application 100 installed on a user device 102. The software application 100 also comprises a server system 104, and the user device 102 is communicatively coupled to the server system 104 via a communication network 106, such as the Internet. The user device 102 is capable of facilitating a user to input data, receive data, and/or transmit data within the software application 100. It will be apparent to a user of ordinary skill in the art that the user may be any personnel using the software application 100, without deviating from the scope of the disclosure. Examples of the user device 102 may include, but are not limited to, a desktop, a notebook, a laptop, a handheld computer, a touch sensitive device, a tablet, a computing device, a smart-phone, and/or a smart watch, etc., or any other suitable device as is known in the art.

The server system 104 is a network of computers, a software framework, or a combination thereof, that may provide a generalized approach to creating the server implementation. More specifically, the server system 104 includes an application server 108 and one or more associated databases 110. The application server 108 and the databases 110 can be integrated as a single unit or can be coupled to each other through a network. Examples of the application server 108 may include, but are not limited to, personal computers, laptops, mini-computers, mainframe computers, any non-transient and tangible machine that can execute a machine-readable code, cloud-based servers, distributed server networks, or a network of computer systems, etc. The application server 108 can be realized through various web-based technologies such as, but not limited to, a Java web-framework, a . NET framework, a personal home page (PHP) framework, or any web-application framework, etc. The server system 104 can be maintained by a storage facility management authority or a third-party entity that facilitates service enablement and resource allocation operations of the software application 100.

The application server 108 is configured to have the logic, instructions, circuitry, interfaces, and/or codes for executing various operations required for communication between the server 108 and the user device 102 and also between the databases 110 and the server 108. The databases 110 are configured to store therein, but are not limited to, data associated with the veterans data, contact data, mental health professionals data, user data, advertisements/services data, etc., from the software application 100. In some embodiments, a set of centralized or distributed network of peripheral memory devices may be interfaced with the server 104, as an example, on a cloud server.

The communication network 106 may include suitable logic, circuitry, and interfaces that may be configured to provide a plurality of network ports and a plurality of communication channels for transmission and reception of data related to operations of various entities (such as the user device 102 and the server system 104) of the software application 100. Each network port may correspond to a virtual address (or a physical machine address) for transmission and reception of the communication data. For example, the virtual address may be an Internet Protocol Version 4 (IPV4) (or an IPV6 address), and the physical address may be a Media Access Control (MAC) address. The communication network 106 may be associated with an application layer for implementation of communication protocols based on one or more communication requests from the user device 102 and the server system 104. The communication data may be transmitted or received via the communication protocols. Examples of the communication protocols may include, but are not limited to, Hypertext Transfer Protocol (HTTP), File Transfer Protocol (FTP), Simple Mail Transfer Protocol (SMTP), Domain Network System (DNS) protocol, Common Management Interface Protocol (CMIP), Transmission Control Protocol and Internet Protocol (TCP/IP), User Datagram Protocol (UDP), Long Term Evolution (LTE) communication protocols, or any combination thereof.

In one embodiment, the communication data may be transmitted or received via at least one communication channel or a plurality of communication channels in the communication network 106. The communication channels may include, but are not limited to, a wireless channel, a wired channel, a combination of wireless and wired channel thereof. The wireless or wired channel may be associated with a data standard which may be defined by one of a Local Area Network (LAN), a Personal Area Network (PAN), a Wireless Local Area Network (WLAN), a Wireless Sensor Network (WSN), Wireless Area Network (WAN), Wireless Wide Area Network (WWAN), a metropolitan area network (MAN), a satellite network, the Internet, a fiber optic network, a coaxial cable network, an infrared (IR) network, a radio frequency (RF) network, and a combination thereof. Embodiments of the present invention are intended to include or otherwise cover any type of communication channel, including known, related art, and/or later developed technologies.

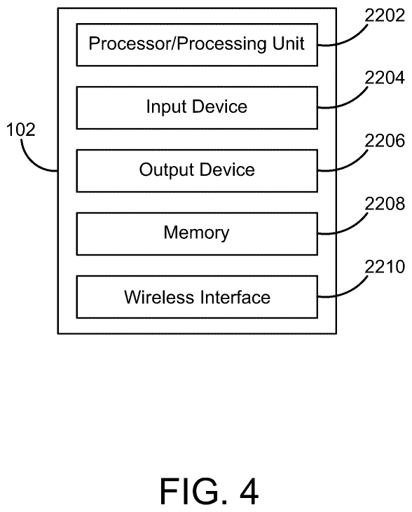

FIG. 4 illustrates an exemplary user device 102 that is used for installing the software application 100 of the present invention. The processing unit 2202 may include suitable logic, instructions, circuitry, interfaces, and/or codes for executing various operations, such as the operations associated with the user device 102, etc. The processing unit 2202 may be configured to control one or more operations executed by the user device 102 in response to the input received at the user device 102 from the user. The processor 2202 executes the computer readable instructions stored in the software application 100. Examples of the processing unit 2202 may include, but are not limited to, an application-specific integrated circuit (ASIC) processor, a reduced instruction set computing (RISC) processor, a complex instruction set computing (CISC) processor, a field-programmable gate array (FPGA), a Programmable Logic Control unit (PLC), etc. Embodiments of the present disclosure are intended to include or otherwise cover any type of processing unit 2202 including known, related art, and/or later developed processing units. The user device 102 can further include one or more computer executable applications configured to be executed by the processing unit 2202. The one or more computer executable applications may include suitable logic, instructions, and/or codes for executing various operations. The one or more computer executable applications may be stored in the memory 2208. The one or more computer executable applications includes the software application 100.

The user device 102 comprises input device(s) 2204, such as a touch input device, a voice input device, etc., for entering data and information. Preferably, the touch interface of the user device 102 is used as the input, and various buttons/tabs shown on the software application 100 are pressed or clicked by the user.

The user device 102 also comprises a memory 2208 used for storing programs (sequences of instructions) or data (i.e., program state information) on a temporary or permanent basis for use in the computer system. Memory 2208 can be configured for short-term storage of information as volatile memory and therefore not retain stored contents if powered off. Examples of volatile memories include random-access memories (RAM), dynamic random-access memories (DRAM), static random-access memories (SRAM), and other forms of volatile memories known in the art. The processor 2202, in combination with one or more of memory 2208, input device(s) 2204, and output device(s) 2206, is utilized to allow users to execute instructions on the software application 100. The connection to a network is provided by wireless interface 2210.

The wireless interface 2210 enables the user device 102 to communicate with the server 104 and other components of the software application 100 over the communication network 106, according to embodiments of the present disclosure. Examples of the communication interface 2210 may include, but are not limited to, a modem, a network interface such as an Ethernet card, a communication port, and/or a Personal Computer Memory Card International Association (PCMCIA) slot and card, an antenna, a radio frequency (RF) transceiver, one or more amplifiers, a tuner, one or more oscillators, a digital signal processor, a coder-decoder (CODEC) chipset, a subscriber identity module (SIM) card, and a local buffer circuit, etc. It will be apparent to a user of ordinary skill in the art that the communication interface 2210 may include any device and/or apparatus capable of providing wireless or wired communications between the user device 102 and the server 104.

Embodiments of the present disclosure take the form of computer-executable instructions, including algorithms executed by a programmable computer. However, the disclosure can be practiced with other computer system configurations as well. Certain aspects of the disclosure can be embodied in a special-purpose computer or data processor that is specifically programmed, configured, or constructed to perform one or more of the computer-executable algorithms described below. Accordingly, the term “computer” as generally used herein refers to any data processor and includes Internet appliances, hand-held devices (including tablets, computers, wearable computers, cellular or mobile phones, multi-processor systems, processor-based or programmable consumer electronics, network computers, minicomputers) and the like.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “veterans assistance application device”, “veterans assistance device”, “software application”, and “device” are interchangeable and refer to the veterans assistance application device 100 of the present invention.

Notwithstanding the foregoing, the veterans assistance application device 100 of the present invention can be of any suitable size and configuration as is known in the art without affecting the overall concept of the invention, provided that it accomplishes the above-stated objectives. One of ordinary skill in the art will appreciate that the veterans assistance application device 100 as shown in FIGS. 1-4 is for illustrative purposes only, and that many other sizes and shapes of the veterans assistance application device 100 are well within the scope of the present disclosure. Although the dimensions of the veterans assistance application device 100 are important design parameters for user convenience, the veterans assistance application device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A veterans assistance application device that provides networking services to combat veteran mental health issues, the veterans assistance application device comprising:
a software application;
wherein the software application is accessible on a smart device;
wherein a user utilizes the software application to allow veterans to communicate and assist one another in dealing with their mental health issues outside of therapy sessions; and further wherein the user utilizes the software application to reconnect and communicate with other veterans from previous deployments or connect with veterans experiencing similar mental health issues.

2. The veterans assistance application device of claim 1, wherein the smart device is a smartphone, tablet, laptop, or a desktop computer.

3. The veterans assistance application device of claim 2, wherein the software application helps veterans build networks to combat mental health issues, such as depression, anxiety, or suicide.

4. The veterans assistance application device of claim 3 further comprising a plurality of indicia.

5. The veterans assistance application device of claim 4, wherein the software application allows a user to create a secure account login.

6. The veterans assistance application device of claim 5, wherein the software application allows a business owner to create a secure account login.

7. The veterans assistance application device of claim 6, wherein the software application allows the business owner to create a profile, create an advertisement offering for sale their services and/or goods, and create a payment page disclosing pricing and fees for the services and/or goods.

8. The veterans assistance application device of claim 7, wherein the software application allows a user to create a profile by uploading identification, such as current ID and military ID.

9. The veterans assistance application device of claim 8, wherein the software application allows the user to click on a specific state to view that state's resources and available mental health services.

10. The veterans assistance application device of claim 9, wherein each state will have a veterans tab and a business tab.

11. The veterans assistance application device of claim 10, wherein the veterans tab will display active veterans and users can then click on a specific veteran they wish to contact.

12. The veterans assistance application device of claim 11, wherein clicking on the specific veteran allows the user to view the specific veteran's profile, and view chat, phone, or camera icons as options to communicate.

13. The veterans assistance application device of claim 12, wherein the business tab will display all businesses with veteran offers and users can then click on a specific business they are interested in.

14. The veterans assistance application device of claim 13, wherein clicking on the specific business will pull up their advertisement, disclosing their offered goods and/or services, and view chat, phone, or camera icons as options to communicate with the specific business.

15. A veterans assistance application device that provides networking services to combat veteran mental health issues, the veterans assistance application device comprising:
a software application;
wherein the software application is accessible on a smart device;
wherein the software application allows a user and a business owner to create a secure account login;
wherein the software application allows the user to click on a specific state to view that state's resources and available mental health services;
wherein each state will have a veterans tab and a business tab;
wherein the veterans tab will display active veterans and users can then click on a specific veteran they wish to contact via chat, phone, or camera icons;
wherein the business tab will display all businesses with veteran offers and users can then click on a specific business they are interested in contacting via chat, phone, or camera icons;
further wherein a user utilizes the software application to allow veterans to communicate and assist one another in dealing with their mental health issues outside of therapy sessions; and
wherein the user utilizes the software application to reconnect and communicate with other veterans from previous deployments or connect with veterans experiencing similar mental health issues.

16. The veterans assistance application device of claim 15, wherein the smart device is a smartphone, tablet, laptop, or a desktop computer.

17. The veterans assistance application device of claim 15, wherein the software application helps veterans build networks to combat mental health issues, such as depression, anxiety, or suicide.

18. The veterans assistance application device of claim 15, wherein users can obtain a subscription to the software application, which allows them to communicate directly with listed veterans and businesses and have full access to all of the software application's services, features, and functionality.

19. The veterans assistance application device of claim 16, wherein the software application includes advertisements and services to earn money for the software application.

20. A method of building veteran networks to combat mental health issues, the method comprising the following steps:
providing a veterans assistance application device comprising a software application accessible on a smartphone;
accessing the software application;
communicating with veterans from previous deployments via the software application;
communicating with veterans experiencing similar mental health issues via the software application;
assisting one another outside of therapy sessions; and
providing advertisements and services to earn money for the software application.
